(19) United States
(12) Patent Application Publication
(10) Pub. No.: US 2024/0228367 A9
(43) Pub. Date: Jul. 11, 2024

(54) GLASS CERAMIC COMPOSITION, PREPARATION METHOD AND APPLICATION THEREOF

(71) Applicant: SHENZHEN YURUCHENG DENTAL MATERIALS CO., LTD., Shenzhen (CN)

(72) Inventors: Zongyu LI, Shenzhen (CN); Wei LIU, Shenzhen (CN); Jian WANG, Shenzhen (CN); Jianjun LIU, Shenzhen (CN); Yu ZHANG, Shenzhen (CN)

(73) Assignee: SHENZHEN YURUCHENG DENTAL MATERIALS CO., LTD., Shenzhen (CN)

(21) Appl. No.: 18/475,632

(22) Filed: Sep. 27, 2023

(30) Foreign Application Priority Data
Oct. 25, 2022 (CN) 202211309304.7

(51) Int. Cl. C03C 10/00; A61C 13/083; C03B 32/02; C03C 4/00; C03C 4/02; C03C 4/12

(52) U.S. Cl. CPC C03C 10/0054; A61C 13/083; C03B 32/02; C03C 4/0021; C03C 4/02; C03C 4/12; C03C 10/00; C03C 2204/00

(57) ABSTRACT

Disclosed are a glass ceramic composition, a preparation method and application thereof. The glass ceramic composition according to the present application includes: 58% to 72% of SiO₂; 0% to 4% of Al₂O₃; 0% to 5% of Na₂O; 3% to 8% of K₂O; 8% to 17% of Li₂O; 2.5% to 5% of P₂O₅, 0% to 2% of MgO, 0% to 2.5% of B₂O₃; 0% to 5% of ZnO; and 0% to 3% of ZrO₂. It is possible to prepare such lithium metasilicate glass ceramic with a crystal phase of lithium metasilicate, a crystallinity of greater than 50%, a grain size of 30 nm to 60 nm and a hardness of 500 kgf/mm² to 590 kgf/mm². The composition does not require an introduction of a high proportion of zirconia content, and can be ground to a thickness of 0.2 mm without edge collapse.

### CROSS-REFERENCE TO RELATED APPLICATIONS

This application claims priority to Chinese Patent Application No. 202211309304.7, filed on Oct. 25, 2022, the entire contents of which are incorporated herein by reference.

### TECHNICAL FIELD

The present application relates to the technical field of dental repair materials, and in particular, to a glass ceramic composition, a preparation method and an application thereof.

### BACKGROUND

Lithium disilicate glass ceramics are widely used in the field of dental restoration due to their excellent mechanical properties and outstanding aesthetic effects. At present, it is mainly applied to veneers, dental bridges, crowns, inlays, implant abutments, etc. Due to the high strength of the lithium disilicate glass ceramics, it is difficult to process and grind them by using computer aided design (CAD)/computer aided manufacturing (CAM), which seriously affects the service life of the bur. In the industry, a matrix glass is often crystallized to generate a lithium metasilicate glass ceramic, then is molded and processed, finally the formed lithium metasilicate glass ceramic is heat treated to obtain a lithium disilicate glass ceramic. Since the strength of the lithium metasilicate glass ceramic is lower than that of the lithium disilicate glass ceramic, molding and processing are relatively easy.

In actual production, due to the accompanying formation of lithium metasilicate and lithium disilicate, uncontrollable formation of lithium disilicate can lead to an increase in the strength of the glass ceramic, which often causes the bur to break and affect grinding efficiency, thereby increasing processing costs. In order to avoid this situation, manufacturers in the industry often produce the lithium metasilicate glass ceramic with low crystallinity by decreasing the crystallization temperature. Although the glass ceramic with low crystallinity can reduce the strength and facilitate grinding, their products have poor toughness due to the high proportion of glass phase, and their edges are prone to cracking when veneers or crowns is processed. In order to solve this problem, some manufacturers in the industry increase the zirconium content in the matrix glass to improve the toughness. However, a small zirconium content is difficult to enhance the toughness. Although a higher zirconium content (mass ratio>10%) can enhance the toughness and improve grinding performance, its requirements for glass melting are too high, and the costs is high. On the other hand, zirconia has a high density and is difficult to melt, which often leads to zirconium deposition and affects the uniformity of the product. Therefore, there is an urgent need for a low-cost glass ceramic that is not prone to edge cracking during grinding.

### SUMMARY

In order to overcome the above-mentioned shortcomings of the related art, the present application provides a glass ceramic composition, a preparation method and an application, which solves the technical problem of edge cracking during grinding in glass ceramic products. The specific technical solutions are as follows.

A glass ceramic composition, calculated by mass percentage, includes: 58% to 72% of SiO₂; 0% to 4% of Al₂O₃; 0% to 5% of Na₂O; 3% to 8% of K₂O; 8% to 17% of Li₂O; 2.5% to 5% of P₂O₅, 0% to 2% of MgO, 0% to 2.5% of B₂O₃; 0% to 5% of ZnO; and 0% to 3% of ZrO₂.

In the glass ceramic composition of the present application, SiO₂ is the main network forming agent, which can stabilize the network structure, constitute the main structure of the glass ceramic, and is also the main component of the crystal phase. In the glass ceramic composition, if the content of SiO₂ is too low, it can lead to changes in the types of crystal phases, and also weaken the overall performance of the glass ceramic. The content of SiO₂ should not be lower than 58 wt %. However, a higher content of SiO₂ can lead to difficulty in melting and forming, and the crystal phase can also form a solid solution of SiO₂. Therefore, based on comprehensive consideration, the present application controls the content of SiO₂ to be 58 wt % to 72 wt %.

In some embodiments, Al₂O₃ is contained, which has a certain effect on the permeability adjustment of the glass ceramic. The higher the content, the better the permeability. However, Al₂O₃ is an extremely refractory oxide, which can quickly increase the high-temperature viscosity of the glass and make it more difficult to clarify and homogenize the glass. The bubble defects are not easy to discharge, therefore the content of Al₂O₃ is controlled to be less than 4 wt %.

The glass ceramic precursor contains alkali metal oxides R₂O, which are mainly Na₂O, K₂O and Li₂O; wherein Na₂O is mainly used to reduce the viscosity of the glass ceramic melt and promote the melting and clarification of the precursor. However, excessive content of Na₂O can increase coefficient of thermal expansion (CTE) and lead to changes in mechanical properties. Therefore, the content of Na₂O is controlled to be 0 wt % to 5 wt %.

K₂O is beneficial to enhance the gloss of the glass ceramic, and its content is not less than 3%. Not limited to theory, it is found through experiments in the present application that a higher content of K₂O can inhibit the formation of lithium disilicate during secondary sintering, which is not conducive to the final conversion of lithium metasilicate, resulting in a decrease in the performance of dental products. Therefore, the content of K₂O is controlled to be 3 wt % to 8 wt %.

Li₂O is the main component of the crystal phase. When the content of Li₂O is too low, the crystal phase cannot be formed, and the minimum content needs to be greater than 8 wt %. However, too high content of Li₂O can cause uncontrolled crystallization, leading to glass crystallization during molding, thereby resulting in defects and affecting subsequent processes.

P₂O₅ can reduce the activated energy of nucleation, facilitate the crystallization of glass, and serve as a nucleating agent for the glass ceramic. If the content of P₂O₅ is too low, the glass is difficult to crystallize. The content of P₂O₅ is at least 2.5 wt %. However, if the content of P₂O₅ is too high, the phase separation of the glass can be severe, which can affect the permeability of the glass ceramic. Therefore, the content of P₂O₅ is at most 5 wt %.

In some embodiments, alkaline earth metal oxide RO is contained, and R²⁺ is Mg²⁺ and Zn²⁺, which can improve the chemical stability and mechanical strength of the glass. In the present application, the content of MgO is limited to be 0% to 2 wt % and ZnO to be 0% to 5 wt %.

B₂O₃ is a network former oxide, which can reduce the high-temperature melting viscosity of the glass, improve the melting characteristics, and facilitate the homogenization of glass components at high temperature. However, a high content of B₂O₃ can easily lead to phase separation and defects in the glass. Therefore, the content of B₂O₃ is controlled to be 0% to 2.5 wt %.

In an embodiment, the mass percentage of each component in the glass ceramic composition satisfies the following relationship:
30%<(SiO₂+Al₂O₃+ZrO₂)—R₂O<60%, where R₂O is a sum of the mass percentage of Na₂O, K₂O and Li₂O;
2%<SiO₂/(Li₂O+P₂O₅)<6%;
7%<Li₂O—P₂O₅<13.5%; and
0.2%<(ZrO₂+ZnO+Na₂O+Al₂O₃)/Li₂O<0.8%.

In an embodiment, when (SiO₂+Al₂O₃+ZrO₂)—R₂O is lower than 30%, the overall viscosity of the glass body is low, which provides an extremely convenient flow environment for the movement of crystalline protons, making it difficult to control the crystallization process, affecting the uniformity of crystallization, and also making it easier to convert lithium metasilicate into lithium disilicate. When (SiO₂+Al₂O₃+ZrO₂)—R₂O is greater than 60%, the overall viscosity becomes larger, and the overall temperature required the crystalline material rises. In order to match the rapid sintering furnace of a denture factory or hospital, it is necessary to ensure that the secondary sintering temperature is controlled below 900° C. Therefore, (SiO₂+Al₂O₃+ZrO₂)—R₂O is controlled to be 30 wt % to 60 wt %.

In an embodiment, in order to maximize the control of the crystallinity of lithium metasilicate, it is necessary to ensure that the ratio of the mass of SiO₂ to the total mass of Li₂O and P₂O₅ is greater than 2; when the ratio is too low, excessive Li₂O and P₂O₅ in the composition can induce the direct generation of lithium disilicate, resulting in a small proportion of the crystal phase of lithium metasilicate; when the ratio is too high, the excessive SiO₂ can form a solid solution of SiO₂ during the secondary sintering (T>800° C.), resulting in an increase in the crystal phase of the final product and affecting the final performance.

Similarly, in order to avoid the formation of Li₃PO₄ crystal phase during the secondary sintering, resulting in an increase in the crystal phase of the final product, Li₂O—P₂O₅ is limited to be 7% to 13.5%.

Since dental products have requirements on the final transmittance of the teeth, a higher transmittance appears too bright and unreal, while a too low transmittance appears burnt and lack luster. For this reason, the ratio of the total mass of ZrO₂, ZnO, Na₂O and Al₂O₃ to the mass of Li₂O is limited to be 0.2% to 0.8%.

In an embodiment, in order to make the color of the glass ceramic product similar to that of natural teeth, the glass ceramic composition further includes colorants and/or fluorescent agents, the content of which is 1% to 6%.

In an embodiment, the colorants and/or fluorescent agents are one or more of TiO₂, La₂O₃, V₂O₅, CeO₂, Er₂O₃, Y₂O₃, Nd₂O₃, Pr₂O₃, Mn₂O₃, NiO and Fe₂O₃.

The present application also provides a preparation method of a glass ceramic composition, as shown in FIG. 4, including the following steps:
S1, weighing and mixing each component of the glass ceramic composition described above according to a ratio to obtain a mixture, and melting and annealing the mixture;
S2, performing crystallization heat treatment, heating to a nucleation temperature of 500° C. to 570° C. at a heating rate of 3° C./min to 10° C./min, keeping the temperature for 5 min to 30 min, then heating to a crystallization temperature of 700° C. to 730° C. at a heating rate of 3° C./min to 5° C./min, and keeping the temperature for 5 min to 20 min; and
S3, lowering the temperature to obtain the glass ceramic composition.

In an embodiment, the melting in step S1 includes: putting the mixture into a crucible, and melting the mixture at a temperature range of 1400° C. to 1500° C. for 2 h to 4 h.

The present application also provides a glass ceramic product prepared according to the above preparation method.

In an embodiment, the crystal phase of the glass ceramic product is lithium metasilicate, and the crystallinity of the glass ceramic composition is greater than 50%.

In an embodiment, the crystal grains of the glass ceramic product are granular, and the average grain size of the crystal grains is 30 nm to 60 nm.

After the glass ceramic product is ground, it can be kept at 830° C. to 850° C. for 5 min to 10 min in a rapid sintering furnace to complete the sintering of the denture.

After secondary sintering, the flexural strength of the glass ceramic product is greater than 350 Mpa.

After secondary sintering, the fracture toughness of the glass ceramic product is greater than 2.2 Mpa·m^{1/2}.

After secondary sintering, the crystal phase of the glass ceramic product has plate-shaped interlocked lithium disilicate, with a crystallinity of greater than 80%.

The present application also provides an application of the above-mentioned glass ceramic composition or the glass ceramic product prepared according to the above-mentioned preparation method in dental materials, such as the preparation of dentures, veneers, inlays, crown repairs, etc.

Compared with the related art, the present application has the following advantages: by optimizing the ratio of the glass ceramic composition and combining with a matching process system, the present application eliminates the need to introduce a high proportion of zirconium oxide content and controls the generation of lithium disilicate to increase the crystallinity of the lithium metasilicate glass ceramic, thereby improving the grinding properties of the glass ceramic composition. The present application can prepare lithium metasilicate glass ceramic with a crystal phase of lithium metasilicate, a crystallinity of greater than 50%, a grain size of 30 nm to 60 nm, and a hardness of 500 kgf/mm² to 590 kgf/mm². The lithium metasilicate glass ceramic does not require the introduction of a high ratio of zirconia content and can be ground to a thickness of 0.2 mm without edge collapse. When the glass ceramic is kept at 830° C. to 850° C. for 5 min to 10 min in a rapid sintering furnace, it is possible to prepare the glass ceramic denture product with the crystal phase as lithium disilicate, a crystallinity of greater than 80%, flexural strength of greater than 350 MPa and fracture toughness of greater than 2.2 Mpa·m^{1/2}. The product of the present application has a short holding time for heat treatment, high grinding efficiency, less damage to the bur, and less edge collapse of the product. It has a very competitive advantage and has a great application prospect.

### BRIEF DESCRIPTION OF THE DRAWINGS

In order to more clearly describe the technical solutions in the embodiments of the present application, a brief introduction will be given to the accompanying drawings required in the description of the embodiments. Obviously, the drawings in the following description are only some embodiments of the present application. For those skilled in the art, other drawings can also be obtained based on these drawings without any creative effort.

FIG. 1 is a scanning electron microscope (SEM) image of a sample before secondary sintering according to Embodiment 2 of the present application.

FIG. 2 is a grinding effect picture of the sample before secondary sintering according to Embodiments 1 to 4 of the present application.

FIG. 3 is a SEM image of the sample after being heated at 840° C. for 5 min in a rapid sintering furnace according to Embodiment 3 of the present application.

FIG. 4 is a flowchart showing a preparation method for a glass ceramic composition according to the present application.

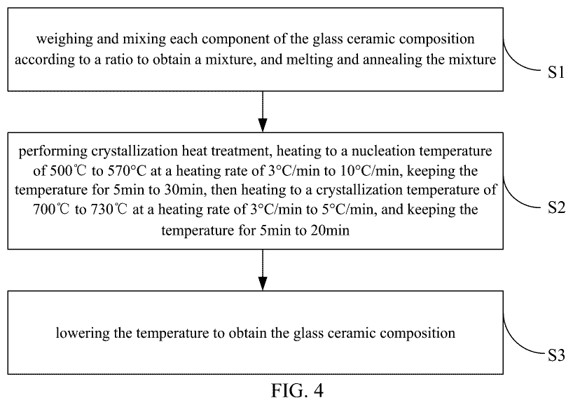

### DETAILED DESCRIPTION OF THE EMBODIMENTS

The technical solutions in the embodiments of the present application will be clearly and completely described below with reference to the accompanying drawings in the embodiments of the present application. It is obvious that the embodiments to be described are only some rather than all of the embodiments of the present application. All other embodiments obtained by persons skilled in the art based on the embodiments of the present application without creative efforts shall fall within the scope of the present application.

Performance testing methods and standards.

Average grain size: measured using a scanning electron microscope (SEM), scanning a surface by the SEM to observe a diameter of particles, adding up the average diameter size of all grain cross-sections and divided by the number of grains in the SEM image.

Crystal phase content: comparing XRD diffraction peaks with the database spectrum to determine the crystal phase, and calculating the proportion of diffraction intensity of the crystal phase in the overall spectrum intensity by a Rietveld method to obtain the crystallinity and amorphous content.

Vickers hardness: GB/T 16534-2009 “Test Method for Room Temperature Hardness of Fine Ceramics”, measured using a Vickers hardness tester, with a loading force of 200 g and a loading time of 10 s.

Grinding effect: using an intelligent tooth carving machine.

Flexural strength: GB 30367-2013 “Dental Ceramic Materials”, tested using a universal testing machine.

Fracture toughness: GB 30367-2013 “Dental Ceramic Materials”, using a universal testing machine and four-point bending test.

Embodiments 1 to 8 are prepared by the following method: first, weighing each component according to a ratio in Table 1 and mixing evenly, putting the uniform mixture into a crucible made of platinum or platinum rhodium, melting within a temperature range of 1450° C. in an electric furnace for 3 h, stirring three times to make it uniform, lowering to an appropriate temperature and casting into a mold, placing the cast glass block into an annealing furnace at 450° C. for annealing, and cooling to a room temperature in the furnace and taking out after the annealing. The glass product after the annealing is placed into a crystallization furnace for crystallization heat treatment to obtain the glass ceramic product. The process conditions of the crystallization heat treatment are shown in Table 2. The test results are shown in Table 3.

TABLE 1
sample composition in Embodiments 1 to 8

| Composition | Embodiment | Embodiment | Embodiment | Embodiment | Embodiment | Embodiment | Embodiment | Embodiment |
|---|---|---|---|---|---|---|---|---|
| (wt %) | 1 | 2 | 3 | 4 | 5 | 6 | 7 | 8 |
| SiO₂ | 68.00 | 59.00 | 70.00 | 71.20 | 67.20 | 62.56 | 69.77 | 67.75 |
| Al₂O₃ | 2.05 | 2.90 | 0.00 | 3.20 | 1.92 | 3.78 | 1.56 | 2.25 |
| Na₂O | 0.80 | 2.20 | 3.80 | 0.50 | 3.20 | 2.54 | 0.00 | 0.30 |
| K₂O | 3.60 | 5.80 | 3.50 | 6.48 | 4.80 | 3.10 | 4.41 | 3.70 |
| Li₂O | 12.30 | 15.25 | 13.78 | 10.25 | 14.15 | 15.23 | 16.20 | 15.50 |
| P₂O₅ | 3.00 | 4.55 | 3.15 | 2.80 | 4.50 | 3.70 | 3.10 | 3.00 |
| ZrO₂ | 1.22 | 2.10 | 0.23 | 0.00 | 1.10 | 0.85 | 0.90 | 2.50 |
| B₂O₃ | 0.50 | 0.50 | 1.00 | 0.00 | 0.20 | 0.00 | 0.20 | 0.20 |
| MgO | 0.20 | 0.10 | 0.37 | 0.00 | 0.18 | 0.68 | 0.00 | 0.00 |
| ZnO | 2.88 | 3.33 | 2.05 | 2.11 | 1.74 | 4.50 | 1.85 | 2.25 |
| TiO₂ | 0.70 | 0.00 | 0.00 | 0.30 | 0.00 | 0.45 | 0.00 | 0.50 |
| V₂O₅ | 1.70 | 2.00 | 0.50 | 0.80 | 0.30 | 0.15 | 0.30 | 0.25 |
| CeO₂ | 2.77 | 1.50 | 1.35 | 2.10 | 1.85 | 2.20 | 1.25 | 1.55 |
| Er₂O₃ | 0.20 | 0.60 | 0.15 | 0.22 | 0.33 | 0.00 | 0.00 | 0.18 |
| Y₂O₃ | 0.00 | 0.00 | 0.02 | 0.03 | 0.00 | 0.01 | 0.00 | 0.00 |
| Nd₂O₃ | 0.00 | 0.00 | 0.00 | 0.01 | 0.00 | 0.00 | 0.00 | 0.00 |
| La₂O₃ | 0.00 | 0.00 | 0.10 | 0.00 | 0.15 | 0.00 | 0.00 | 0.00 |
| Mn₂O₃ | 0.00 | 0.05 | 0.00 | 0.00 | 0.00 | 0.00 | 0.05 | 0.05 |
| NiO | 0.00 | 0.02 | 0.00 | 0.00 | 0.01 | 0.00 | 0.00 | 0.00 |
| Fe₂O₃ | 0.03 | 0.00 | 0.00 | 0.00 | 0.00 | 0.25 | 0.41 | 0.02 |
| Pr₂O₃ | 0.05 | 0.10 | 0.00 | 0.00 | 0.80 | 0.00 | 0.00 | 0.00 |
| (SiO₂ + Al₂O₃ + | 54.57 | 40.75 | 49.15 | 57.17 | 48.07 | 46.32 | 51.62 | 53.00 |
| ZrO₂) − R₂O | | | | | | | | |
| SiO₂/(Li₂O + | 4.44 | 2.98 | 4.13 | 5.46 | 3.60 | 3.30 | 3.62 | 3.66 |
| P₂O₅) | | | | | | | | |
| Li₂O − P₂O₅ | 9.30 | 10.70 | 10.63 | 7.45 | 9.65 | 11.53 | 13.10 | 12.50 |
| content of | 5.45 | 4.27 | 2.12 | 3.46 | 3.44 | 3.06 | 2.01 | 2.55 |
| colorant | | | | | | | | |
| (ZrO₂ + ZnO + | 0.57 | 0.69 | 0.44 | 0.57 | 0.56 | 0.77 | 0.27 | 0.47 |
| Na₂O + Al₂O₃)/Li₂O | | | | | | | | |

TABLE 2
heat treatment process conditions in Embodiment 1 to 8

| heat treatment | Embodiment | Embodiment | Embodiment | Embodiment | Embodiment | Embodiment | Embodiment | Embodiment |
|---|---|---|---|---|---|---|---|---|
| process | 1 | 2 | 3 | 4 | 5 | 6 | 7 | 8 |
| nucleation | 520 | 550 | 560 | 530 | 510 | 520 | 550 | 570 |
| temperature (° C.) | | | | | | | | |
| nucleation time | 10 | 20 | 20 | 10 | 30 | 25 | 10 | 5 |
| (min) | | | | | | | | |
| crystallization | 700 | 720 | 720 | 710 | 730 | 710 | 730 | 700 |
| temperature (° C.) | | | | | | | | |
| crystallization | 20 | 10 | 10 | 20 | 5 | 10 | 10 | 10 |
| time (min) | | | | | | | | |

| | |
|---|---|
| secondary | 840 |
| sintering | |
| temperature (° C.) | |
| secondary | 5 |
| sintering time | |
| (min) | |

TABLE 3
test results in Embodiment 1 to 8

| | Embodiment | Embodiment | Embodiment | Embodiment | Embodiment | Embodiment | Embodiment | Embodiment |
|---|---|---|---|---|---|---|---|---|
| test item | 1 | 2 | 3 | 4 | 5 | 6 | 7 | 8 |
| crystal phase | lithium metasilicate | | | | | | | |
| crystallinity | >50% | | | | | | | |
| grain size (nm) | 42 | 48 | 45 | 50 | 50 | 46 | 55 | 32 |
| Vickers | 510 | 522 | 514 | 578 | 565 | 570 | 582 | 550 |
| hardness | | | | | | | | |
| (kgf/mm²) | | | | | | | | |
| grinding effect | 0.2, no edge collapse | | | | | | | |
| (mm) | | | | | | | | |
| flexural | 355 | 380 | 365 | 408 | 378 | 369 | 388 | 393 |
| strength after | | | | | | | | |
| secondary | | | | | | | | |
| sintering | | | | | | | | |
| (MPa) | | | | | | | | |
| fracture | 2.3 | 2.4 | 2.5 | 2.8 | 2.6 | 2.5 | 2.7 | 2.8 |
| toughness after | | | | | | | | |
| secondary | | | | | | | | |
| sintering | | | | | | | | |
| (2 Mpa · m^{1/2}) | | | | | | | | |

| | |
|---|---|
| crystallinity | >80% |
| after secondary | |
| sintering | |

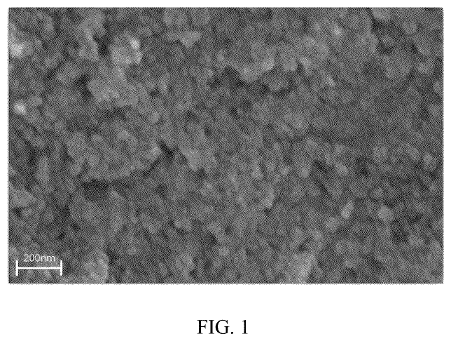

FIG. 1 is a SEM image of the sample before secondary sintering according to Embodiment 2, with the average grain size of 30 nm to 60 nm.

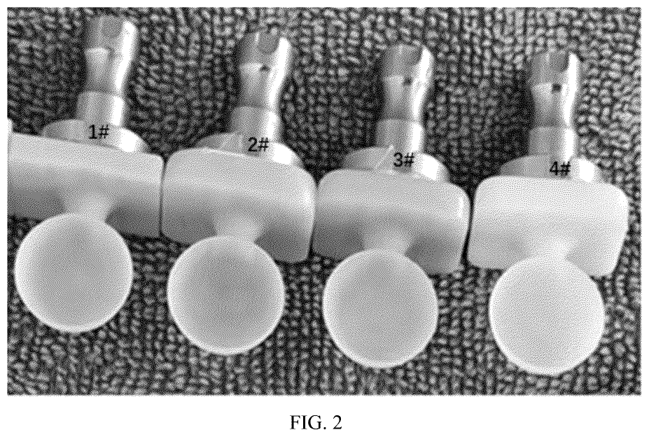

1 # to 4 # in FIG. 2 show the grinding effect of samples before secondary sintering according to Embodiments 1 to 4, with a thickness of an edge wall of 0.2 mm.

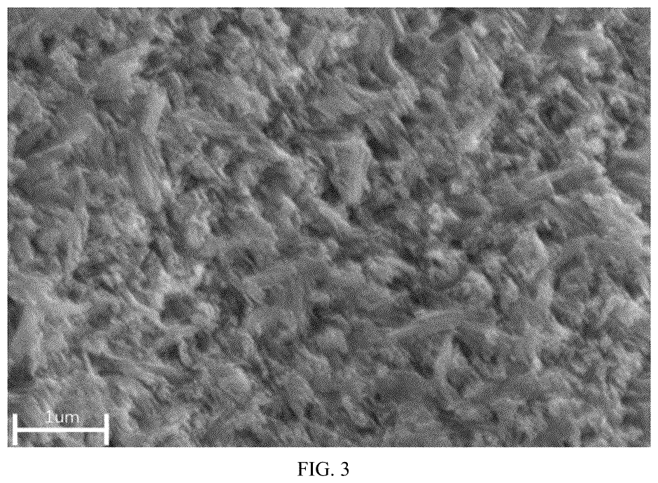

FIG. 3 is a SEM image of the sample after being kept in a rapid sintering furnace at 840° C. for 5 min according to Example 3, with a crystal form of plate-like.

### Comparative Example 1

The difference between Comparative Example 1 and Embodiment 1 is only in the nucleation temperature. The nucleation temperature in Comparative Example 1 is 450° C. It is measured that the crystal phase of the sample in Comparative Example 1 contains very little lithium metasilicate, the crystallinity is 38%, the grain size is 52 nm, the Vickers hardness is 446 kgf/mm², teeth appear edge collapse during grinding a thickness of 0.2 mm, the flexural strength after the second sintering is 306 MPa, the fracture toughness after the second sintering is 1.5 Mpa·m^{1/2}, and the crystallinity after the second sintering is 58%.

### Comparative Example 2

The difference between Comparative Example 2 and Embodiment 1 is only in the nucleation temperature. The nucleation temperature of Comparative Example 2 is 600° C. It is measured that the crystal phase of the sample in Comparative Example 2 is lithium metasilicate and lithium disilicate, the crystallinity is 62%, the grain size is 30 nm, and the Vickers hardness is 635 kgf/mm². The hardness is too high, a bur breaks during grinding, which affects the life of the bur. The grinding effect is 0.2 mm, the flexural strength after the second sintering is 377 MPa, the fracture toughness after the secondary sintering is 2.3 Mpa·m^{1/2}, and the crystallinity after the secondary sintering is greater than 80%.

### Comparative Example 3

The difference between Comparative Example 3 and Embodiment 1 is only in the crystallization temperature. The crystallization temperature of Comparative Example 3 is 650° C. It is measured that the crystal phase of the sample in Comparative Example 3 is lithium metasilicate, the crystallinity is 43%, the grain size is 37 nm, the Vickers hardness is 487 kgf/mm², the teeth appear edge collapse during grinding a thickness of 0.2 mm, the flexural strength after the secondary sintering is 343 MPa, the fracture toughness after the secondary sintering is 1.95 Mpa·m^{1/2}, and the crystallization after the secondary sintering is 76%.

### Comparative Example 4

The difference between Comparative Example 4 and Embodiment 1 is only in the crystallization temperature. The crystallization temperature of Comparative Example 4 is 750° C. It is measured that the crystal phase of the sample in Comparative Example 4 is lithium metasilicate and lithium disilicate, the crystallinity is 72%, the grain size is 65 nm, the Vickers hardness is 667 kgf/mm². The hardness is too high, the bur breaks during grinding to a thickness of 0.2 mm, which affects the life of the bur. The flexural strength after the second sintering is 382 MPa, the fracture toughness after the second sintering is 1.3 Mpa·m^{1/2}, and the crystallinity after the secondary sintering is greater than 80%.

The above are only some embodiments of the present application and are not intended to limit the present application. Any modifications, equivalent substitutions and improvements made within the spirit and principles of the present application shall be included in the scope of the present application.

What is claimed is:

1. A glass ceramic composition, calculated by mass percentage, comprising:
58% to 72% of SiO₂; 0% to 4% of Al₂O₃; 0% to 5% of Na₂O; 3% to 8% of K₂O; 8% to 17% of Li₂O; 2.5% to 5% of P₂O₅, 0% to 2% of MgO, 0% to 2.5% of B₂O₃; 0% to 5% of ZnO; and 0% to 3% of ZrO₂.

2. The glass ceramic composition according to claim 1, wherein the mass percentage of each component in the glass ceramic composition satisfies the following relationship:
30%<(SiO₂+Al₂O₃+ZrO₂)—R₂O<60%,
where R₂O is a sum of the mass percentage of Na₂O, K₂O and Li₂O.

3. The glass ceramic composition according to claim 1, wherein the mass percentage of each component in the glass ceramic composition satisfies the following relationship:
2%<SiO₂/(Li₂O+P₂O₅)<6%.

4. The glass ceramic composition according to claim 1, wherein the mass percentage of each component in the glass ceramic composition satisfies the following relationship:
7%<Li₂O—P₂O₅<13.5%.

5. The glass ceramic composition according to claim 1, wherein the mass percentage of each component in the glass ceramic composition satisfies the following relationship:
0.2%<(ZrO₂+ZnO+Na₂O±Al₂O₃)/Li₂O<0.8%.

6. The glass ceramic composition according to claim 1, further comprising 1 wt % to 6 wt % colorant and/or fluorescent agent.

7. The glass ceramic composition according to claim 1, wherein a crystal phase of the glass ceramic composition is lithium metasilicate, and a crystallinity of the glass ceramic composition is greater than 50%.

8. A preparation method of a glass ceramic composition, comprising the following steps:
weighing and mixing each component according to a ratio of the glass ceramic composition described in claim 1 to obtain a mixture, and melting and annealing the mixture;
performing crystallization heat treatment, heating to a nucleation temperature of 500° C. to 570° C. at a heating rate of 3° C./min to 10° C./min, keeping the temperature for 5 min to 30 min, then heating to a crystallization temperature of 700° C. to 730° C. at a heating rate of 3° C./min to 5° C./min, and keeping the temperature for 5 min to 20 min; and
lowering the temperature to obtain the glass-ceramic composition.

9. The preparation method of the glass ceramic composition according to claim 8, wherein melting the mixture comprises: putting the mixture into a crucible, and melting the mixture at a temperature range of 1400° C. to 1500° C. for 2 h to 4 h.

10. An application of the glass ceramic composition according to claim 1 in dental materials.
